(19) United States
(12) Patent Application Publication
(10) Pub. No.: US 2004/0071760 A1
(43) Pub. Date: Apr. 15, 2004

(54) Therapeutic film forming composition and treatment system therefor

(76) Inventors: Israel Dvoretzky, Hamden, CT (US); John E Kuleza, Berlin, CT (US)

Correspondence Address: Melvin I Stoltz, 51 Cherry Street, Milford, CT, 06460, US

(21) Appl. No.: 10/250,836

(22) PCT Filed: Jan. 7, 2002
(86) PCT No.: PCT/US02/00282

(51) Int. Cl. A61K 9/70; A61K 9/14

(52) U.S. Cl. 424/449; 424/486; 424/488

(57) ABSTRACT

By providing a film forming composition incorporationg one or more therapeutic substances for application to nails and/or skin surfaces which can be employed independently or, if desired, in combination with an easily employed holding or support member for delivering heat directly to the application site an easily employed, convenient, consumer-oriented treatment system is achieved for treating nails and/or skin surfaces for a wide variety of medical problems. The treatment system of the present invention possesses broad applicability for a wide range of medical conditions, including the mumerous diseases, disorders, and medical problems, all of which are capable of being treated using the present invention. In particular, diseases, disorders, and medical conditionss which include, but are not limited to, psoriasis, skin cancers, warts, leishmaniasis, mycobacteria, and granuloma annulare can be specifically treated or improved due to the efficacy of the present invention and, when employed, the efficacy of heat penetration in treating these disorders.

### TECHNICAL FIELD

This invention relates to diseases found in and beneath fingernails, toe-nails and on skin surfaces and, more particularly, to a fully integrated treatment system for delivering drug therapy to diseases affecting nails and skin surfaces.

### BACKGROUND ART

Diseases which affect the skin surfaces and/or the fingernails and/or toenails of individuals are extremely common and often create substantial difficulties for the individuals who are effected. As detailed herein, a wide variety of the diseases have been found to afflict individuals in this manner, with no realistically effective remedy being available.

In order to recognize the difficulties encountered in this area, it is important to understand the unique structure of nails. As is well known, the nail is a unique keratinous appendage that is produced by the germinative epithelium of the matrix. The construction of a typical nail is similar to the construction of the skin surface in which the basal epidermal cells produce the stratum corneum. However, the nail has a harder keratin than the skin because of the higher concentration of sulfur matrix protein.

The nail grows continuously without a resting phase by adding new cells from the matrix. The epithelial components of the nail unit include the proximal nail fold that is similar to the skin or slightly acanthotic, with a granular layer having active mitotic activity. The horny end product is the cuticle. The matrix is acanthotic without a granular layer with very inactive mitotic activity. The horny end product is the nail plate. The nail bed is very flat without a granular layer with very inactive mitotic activity and only a few horny cells added to the underside of the nail plate as it moves distally. Finally, the hyponychium is acanthotic, with a granular layer having active mitotic activity, with only a few horny cells added to the underside of the nail plate distally, similar to the cuticle.

A wide variety of problems can be incurred either in the nail itself or below the nail surface in treating nails and diseases of the nail. In order to fully understand the magnitude of this problem, the following tables are provided as representative sample listings of various diseases, disorders, and medical problems that can be experienced and are in need of effective treatment:

TABLE A
Dermatological Diseases that Affect the Nail

Psoriasis/pustular psoriasis
Lichen Planus
Idiopathic Atrophy of the nail
Lichen Nitidus
Lichen Striatus
Inflanimatory Linear Verrucous Epidermal News
Alopecia Areata
Twenty-Nail Dystrophy (Trachyonychia)
Eczema with nail involvement
Pornpholyx with nail involvement
Parakeratosis pustalosa
Pemphigus Vulgaris
Bullous Pemphigoid
Acquired Epidermolysis Bullosa
Erythema Multiforme with nail involvement
Darier's Disease
Pityriasis Rubra Pilaris
Palmoplantar Keratoderma
Bazex Syndryome

TABLE B
Nail Involvement in Systemic Diseases

Onycholysis which is the third most common nail disorder seen in
dermatology, after onychomycosis and warts.
Chromonychia
Hemorrhagic condition
Onychomadesis (complete onyholysysis or spontaneous nail shedding)
Hapalonychia (soft nail)
Onychorrhexis (senile, old nail)
Trachyonychia

TABLE C
Tumors of the Nails
Epidermal Tumors

Benign
Subungual corn
Wart
Cysts (Epidermal Inclusion/Epidermoid cyst)
Onychomatricoma
Melanocytic Tumors
Glomus Tumor
Myxoid Cyst
Pyogenic Granuloma
Malignant
Bowen's disease/squamous cell carcinoma
Maligant Melonoma

Dermal Tumors

Benign
Fibroma
Vascular Tumors
Glomus Tumor
Pyogenic Granuloma
Splinter Hemorrhages
Subungeal Tumors
Subungeal exostosis
Subungeal Hematomas
Pigmented Disorders
Hypo/Hypermelanosis/melanonychia
Infections
Fungal infections (dermatophytes, yeast, deep fungus)
Bacterial infections (gram positive, gram negative bacteria, etc.)
Viral Infections (herpes simplex, warts, etc.)
Other Infections (Atypical Mycobacteria, sporotrichosis,
leishmaniasis etc.)
Mixed Infection (paronichia)

TABLE D
Congenital Keratinizing Disorders

Darier-White Disease
Pachonichia Congenita
Congenital Onychodysplasia
Epidermolysis bullosa
Twenty-Nail Dystrophy
Pityriasis Rubra Pilaris
Lamellar Ichthyosis
Epidermolytic Hyperkeratosis
Porokeratosis
Palmoplantar keratoderma
Periodic Shedding of the the nails
Ectodermal Dysplasia
Rothmund-Thompson Syndrome
Dyskeratosis Congenita
Acrodermatitis enteropatica
Goldtz' Syndrome
Kid Syndrome

TABLE E
Nail Changes/Disorders Associated with Aging

Changes
Discoloration
Contour/Shape
Surface abnormality
Thickness
Decreased Linear Growth
Disorders
Brittle Nails
Onychodystrophy-exogenous
Onychauxis
Onychoclavus (subungeal corn)
Onychocryptosis (Ingrown nail)
Onychogryphosis
Splinter Hemorrhages
Subungeal Hematoma
Nail Infection

One of the areas that has proven to be extremely difficult for individuals to obtain an effective remedy is found in the treatment of psoriasis, particularly psoriasis associated with fingernails or toenails. It is estimated that there are more than 6.4 million cases of psoriasis in the United States, affecting about 2% to 3% of the general American population. According to the National Psoriasis Foundation, about 200,000 new cases are diagnosed every year, with an annual total outpatient cost of between about $1.6 in $3.2 billion being incurred. Furthermore, it is estimated that 56 million hours of work are lost each year by patients with psoriasis.

Psoriasis of the nails is very common and has been found in up to 50% of all patients. The fingernails seem to be affected more often than toenails, which presents an additional problem since patients are more likely to complain about nail changes which are readily visible. As a result, the effects of psoriatic nail disease are both physical and psychological.

The clinical signs of psoriasis of the nails are dependent upon the anatomic sites of the nail unit that is involved. The defects vary greatly and include pitting of one or more nails, discoloration, thickening, and onycholysis (separation from the nail bed). The effect of onycholysis can also involve the underlying subungual build-up similar to onychomycoses. Other changes commonly found are nail plate dystrophy, Mee's lines (transverse white lines in the nail plate), Muehrcke's lines (transverse white lines due to abnormal vascular pattern in the nail bed visible through the nail plate), splitting of the nail plate, Beau's lines (transverse furrow in the nail plate generally due to intermittent matrix psoriasis), splinter hemorrhage, and subungual hyperkerertosis.

The objective of treating psoriatic nail disease is to improve the physical and physchosocial debilitation associated with nail dystrophy. Unfortunately, no treatment presently in use is capable of curing psoriatic nails. Although numerous attempts and treatment modalities presently exist in the prior art, there is no specific consistently effective treatment for nail psoriasis.

Many modalities have been used, including topical, intralesional, and systemic modalities, radiation therapy, etc. However, most of these prior art treatment modalities had substantial risks and complications, including systemic toxicity, post-radiation complications, atrophy of the surrounding skin, pain, etc. In addition, nail lacquers have been proposed in various prior art formulations for application to the nail for treating psoriasis, as well as many other nail disorders. However, these prior art nail lacquers have also proven to be ineffective in resolving the nail diseases for which they were intended. As a result, a long-felt need has continued to exist for a safe, effective treatment system for nails and nail diseases, with no satisfactory proposal being found until the present invention that will be easy to use, safe, and cost efficient.

Therefore, it is a principal object of the present invention to provide a treatment system for humans which is capable of being conveniently and easily employed on any desired surface of a human, including the nail surface and skin surface, providing a quick drying film, which incorporates desired therapeutic agents for treating various medical problems.

Another object of the present invention is to provide a highly effective treatment system having the characteristics featured detailed above which also employs a heat gradient to further enhance the delivery of the therapeutic agents.

Another object of the present invention is to provide a highly effective treatment system having the characteristics featured detailed above which is capable of being easily and effectively employed for treating a wide variety of medical conditions.

Another object of the present invention is to provide a highly effective treatment system having the characteristics featured detailed above which also incorporates a holding and supporting member constructed or cooperation with the film forming composition and the heat producing member to assure ease of application and use.

Other and more specific objects will in part be obvious and will in part appear hereinafter.

### DETAILED DESCRIPTION

By employing in the present invention, all of the difficulties and drawbacks found in prior art compositions, systems, methods and procedures are eliminated, and a local, easily employed, convenient, consumer-oriented treatment system is achieved for treating nails and/or skin surfaces having a wide variety of medical problems. In the present invention, a treatment system is attained which comprises a film forming composition incorporating one or more therapeutic substances for application to nails and/or skin surfaces which can be employed independently or, if desired, in combination with an easily employed holding or support member for delivering heat directly to the application site.

The treatment system of the present invention possesses broad applicability for a wide range of medical conditions. In particular, the numerous diseases, disorders, and medical problems detailed above are all capable of being treated using the present invention. In particular, diseases, disorders, and medical conditions including, but not limited to psoriasis, skin cancers, warts, leishmaniasis, mycobacteria, and granuloma annulare can be specifically treated or improved due to the efficacy of the present invention and, when employed, the efficacy of heat penetration in treating these disorders.

In this regard, it has been established that the application of heat slows, inhibits, or reverses metabolic processes, immunological processes, or biological conditions or processes which depend upon heat or are affected by heat. As a result, numerous medical conditions are effectively treated by employing the therapeutic or drug bearing film forming compositions detailed herein in combination with a heating source, in accordance with the embodiment of this invention which comprises the integrated treatment system.

It has also been found that the use of heat produces a positive, synergistic effect on the targeted, controlled delivery of predetermined amounts of drugs, and/or penetration enhancing agents. In this regard, the topical use of the drugs and/or penetration enhancing agents for the treatment of the nail diseases, disorders, and medical conditions and/or skin and subcutaneous surfaces, as fully detailed above, are improved or effectively treated by the use of the present invention. Furthermore, the delivery of drugs and/or penetration enhancing agents to the nail, or through the nail to the underlying tissue, for the purpose of achieving a non-oral and/or non-parenteral, systemic, transdermal delivery is effective on its own and is further enhanced by the presence of a controlled heat gradient.

Generally, the treatment system of the present invention is detailed below in association with its application to nails and the underlying tissue associated with nails. However, the present invention has equal applicability to diseases, disorders, and medical problems found on skin surfaces which are unassociated with nails. Consequently, it is to be understood that the present invention and the disclosure provided herein is equally applicable to use on any skin surface. Consequently, the detailed use and application to the nails is provided for exemplary purposes only and, as a result, the present invention is intended to encompass application and use on both nails and skin surfaces.

One principal component of the present invention is a film forming composition which is specifically formulated for delivering one or more therapeutic substances to the site on which it is applied. While usable on any surface of the human body, the film forming position is particularly usable on nails for treating any desired nail disease.

In addition to comprising one or more therapeutic substances, the film forming composition of this invention also preferably comprises a film former, a plasticizer, and urea, all of which are in a volatile carrier. By referring to Table I, the formulation ranges for each ingredient are readily apparent. In this Table, both the overall usable ranges and preferred ranges are provided.

TABLE I

| Ingredient | Overall Range (% w/v) | Preferred Range (% w/v) |
|---|---|---|
| Film Former | 0.5-25 | 5-10 |
| Plasticizer | 0.5-25 | 1-5 |
| Urea | 0.5-20 | 1-6 |
| Therapeutic | Required Effective Amt. | Required Effective Amt |
| Substances | | |
| Solvent/Volatile | 40-90 | 50-80 |
| Carrier | | |

In forming the composition of the present invention, any desired film former may be employed. However, in the preferred composition, the film former comprises at least one selected from the group consisting of polyvinyl acetate, mixed polymers of vinyl acetate and acrylic acid, mixed polymers of (meth) acrylic acid and (meth) acrylate esters, polyvinyl acetate, polyvinyl butyryl, polyvinyl alcohols, cellulose derivatives such as cellulose acetate phthalate, cellulose acetate butyrate, cellulose acetate propionate, cellulose nitrate, cellulose sulfate, ethylcellulose, and cellulose acetate. In this regard, one preferred film former is ammonio methacrylate copolymer (II) sold under the brand name “Eudragit RL-100” by Rohm Pharma GmbH, Weiterstadt, Germany.

In addition, any desired plasticizers may be employed. However, in the preferred composition, the plasticizer composition comprises as least one selected from the group consisting of triacetin, polyhydric alcohols such as propylene glycol and butylene glycol, castor oil, camphor and phthalates. In this regard, one preferred plasticizer comprises dibutyl phthalate, sold under the brand name “Eastman DBT Plasticizer” by Eastman Chemicals.

In formulating the compositions of the present invention, any suitable solvent or volatile carrier may be employed. However, it has been found that the solvent/volatile carrier preferably comprises one or more selected from the group consisting of alcohols (such as ethanol), ketones (such as acetone), ethers (such as chloroform), aromatic hydrocarbons (such as toluene), esters (such as ethyl acetate), and water.

In selecting or formulating a combination of ingredients for the solvent/volatile carrier, it has been found that the preferred compound or compounds should act as a solvent for the carrier and at an evaporation rate which is slow enough to allow a complete application of the active ingredients to nail or skin surfaces, as well as being fast enough to prevent rub-off of the composition in the event the nail or skin surface comes into contact with another surface.

One preferred formulation for the therapeutic film forming composition is detailed in Table II. In forming this preferred composition, the film former namely Eudragit RL-100, is first dissolved in the acetone/ethanol/water carrier. After a clear solution is formed, the other ingredients are added and intermixed therein until completely dissolved. In addition, the desired therapeutic agents are also added and intermixed therewith to form the desired therapeutic film forming composition for application to the nails or skin.

TABLE II

| INGREDIENT | QUANTITY |
|---|---|
| Clobetasol propionate | 0.05% w/v |
| Urea | 3% w/v |
| Dibutyl phthalate | 1.5% w/v |
| Eudragit RL-100 | 7% w/v |
| Ethanol | 70% w/v |
| Water | q.s. to 100% |

In completing the therapeutic film forming composition of this invention, one or more therapeutic substances are incorporated into the compositions in sufficient quantity to provide the effect desired. In general, the therapeutic substance employed comprises one or more selected from the group consisting of corticosteroids, chemotherapeutic agents, anesthetics, antihistamines, anti-bacterials, anti-parasitics, anti-viral agents, anti-oxidants, tar, anthralines, immunomodulators, keratolytics, and anti-neoplastics. Although a wide variety of chemical products come within the scope of these medications, the following list provides typical compositions that are effectively employed as a part of the treatment system of the present invention. However, this listing is provided for exemplary purposes only, and is not intended to limit the present invention thereto.

In this regard, corticosteroids may comprise one or more selected from the group consisting of hydrocortisone, triamcinolone, betamethasone, and any other steroids commonly used in topical applications to the skin; chemothera-peutic agents may comprise one or more selected from the group consisting of 5FU, Bleomycin, methotrexate, cytotoxic agents; anesthetics may comprise one or more selected from the group consisting of lidocaine, prilocaine, and pramoxine, antihistamines may comprise one or more selected from the group consisting of diphenhydramine and its salts; or doxepin, anti-bacterials may comprise one or more selected from the group consisting of gentamicin, tetracycline, erythromycin, and clindamycin; anoxicillin, anti-parasitics may comprise one or more selected from the group consisting of metronidazole, permethrin, and crotamiton; anti-virals may comprise one or more selected from the group consisting of acyclovir; antioxidants may comprise one or more selected from the group consisting of ascorbic acid and tocopherol; immunomodulators may comprise one or more selected from the group consisting of imiquimod and beta glucan; keratolytics may comprise one or more selected from the group consisting of salicylic acid; and anti-neoplastics may comprise one or more selected from the group consisting of cytotoxic agents and immunomodulators.

Furthermore, as detailed herein, it has also been discovered that the use of a heat gradient, as provided by one of the alternate embodiments of the present invention, is effective in causing enhancing agents to be more efficiently employed. In this regard, as is fully detailed below, enhancing agents which particularly benefit from the use of this embodiment of the present invention comprise one or more selected from the group consisting of solvents, surfactants, ethers, esters, fatty acid glycerides, urea, oleates, liposomes, retinoids, and occlusive compounds.

As discussed above, the present invention comprises the use of a therapeutic film forming composition independently in one embodiment, and also comprises the use of a heat delivery member which is employed in combination with the therapeutic film forming composition. In the use of the present invention on a nail and/or skin surfaces, the therapeutic film forming composition is applied to the nail and/or skin surface. Once the film layer is established, the heat delivery member, when employing this embodiment, is placed over the nail to provide a continuous heat gradient directly to the affected nail and/or skin surface.

As stated above, the application of heat has been found to enhance the delivery and efficacy of many therapeutic substances. When employing this embodiment, heat delivery is preferably achieved by employing a heat delivery pad used in combination with a holding member. In the preferred construction, an exothermic pad or heat delivery patch is employed for the heat source. Exothermic pads or heat delivery patches have been previously developed and typically comprise a porous film or pad of woven or non-woven material incorporating chemicals which will react exothermically to generate heat in the presence of oxygen. Although any desired chemicals can be employed, exothermic pads or heat delivery patches typically contain moxa or a mixture of iron powder, activated charcoal, wood fibers, water and salt. Alternatively, a mixture of alkaline sulfides and iron carbide are employed with the chemicals stored in an inert, oxygen-free, chamber to prevent exposure to oxygen prior to use. In addition, the pores of the pad are of sufficient size to assure that the required air flow is achieved. If desired, any alternate heat producing pad, product, or construction can be employed.

Prior to use, the patch/pad is typically sealed within a pouch which is odorless or formed with an inert gas, such as nitrogen. As long as the patch/pad remains in the sealed container until use, no chemical reaction takes place. However, once the pouch is open, the presence of the oxygen in the air causes the chemicals to react and the desired exothermic reaction is produced.

In this embodiment of the present invention, the exothermic pad or heat delivery patch is separately employed by having the user place the pad or patch on the desired site where heat treatment is being sought. If desired, the heat delivery patch or exothermic pad is separately secured to the desired site by adhesive means associated therewith.

Once the patch/pad is secured in the precisely desired location, the holding member of the treatment system of the present invention is affixed to the desired location, peripherally surrounding and securely maintaining the heating pad or exothermic patch in the desired location, assisting in regulating and controlling the heat level and air transmission to the exothermic pad or heat delivery patch. In this way, the desired, controlled heat delivery or heat gradient is realized, enabling a broad range of medical conditions to be effectively treated. In addition to providing controlled heat delivery for the direct treatment of certain medical conditions, the use of a heat gradient as provided by this embodiment of the fully integrated treatment system of the present invention is effective, improving and enhancing the penetration of the systemic and topical medications incorporated in the therapeutic film forming composition.

As is evident from the foregoing, a wide variety of broadly diverse medical conditions are effectively and efficiently treated using the treatment system of the present invention. As detailed above, one embodiment of the treatment system of the present invention incorporates a heat delivery patch or exothermic pad in combination with a holding member which maintains the heat delivery patch or exothermic pad in a precisely desired location where the particular medical problem is manifested or where heat delivery or a heat gradient is desired. In addition, nail or skin penetration enhancing agents may be integrated into the treatment system of this invention for further enhancement of its efficacy.

One area in which the use of heat has been widely documented is in the treatment of warts. In this regard, heat treatment for warts is fully disclosed in Dvoretzky, U.S. Pat. No. 5,053,024. However, the use of heat in treating other skin disorders or medical conditions as well as the use of heat for assisting in the transmission and/or absorption of medicines and nail/skin penetration enhancing agents is generally unknown and represents an advance in this technological area, particularly in combination with a therapeutic film forming composition.

By employing this embodiment of the treatment system of the present invention, which comprises a therapeutic film forming composition in combination with a holding member and a heat delivery patch or exothermic pad, the temperature of the nail or skin is elevated and maintained between about 39° to 45° C., for periods of time ranging between about 1 and 10 hours. In addition, by employing the present invention, the heat level to which the nail or skin is exposed is maintained in a small, controlled range, which represents the optimum heat exposure for treating the medical condition and delivering the precisely desired medicines. As a result, optimum performance and treatment is realized.

In accordance with the present invention, the support member is specifically designed for being quickly and easily mountable to a part of the body, such as a finger, in peripheral, surrounding engagement with the nail being treated, and remain in the precisely desired location in cooperating association with the film forming composition and the heat delivery patch or exothermic pad. Furthermore, the support member of the present invention is also specifically constructed from material particularly designed for cooperating with a heat delivery patch or exothermic pad to synergistically interact therewith for assuring that the precisely required temperature levels are maintained and optimum oxygen flow is realized for the precisely desired period of time.

The support member forming a component of the holding system of the present invention may be constructed in a variety of alternate configurations for providing the desired, quicky, easy, secure mounting thereof directly to any finger or toe, or any other portion of the human anatomy where treatment is likely to be needed. In this regard, one embodiment of the holding system of the present invention comprises a continuous, substantially cylindrically shaped tube member. By employing a cylindrically shaped member, the holding system of the present invention is able to be easily mounted onto the hands or feet of the user, peripherally surrounding the nail to be treated, securely maintaining and cooperating with the pre-mounted exothermic pad or heat delivery patch in the precisely desired location.

One feature of the present invention is the material employed for forming the treatment system of this invention. In this regard, most woven or non-woven materials are capable of being employed for forming the holding member. However, it has been found that the preferred material comprises an elastomer in its entirety or formed therein, in order to provide flexibility in easily mounting and securing the holding system in place. In one preferred construction, the elastomer is integrally formed as a part of the material, such as a thermoplastic elastomer. In this regard, the preferred thermoplastic material comprises a foamed thermoplastic elastomer selected from the group consisting of polyurethane, polyolefins, polybutylenes, polyethylenes, polyesters, ethylene-propylene rubbers, polypropylenes, silicones, and vinyl based resins.

By employing a foamed thermoplastic elastomer, a precisely desired combination of closed cells and open cells is obtained, with the ratio of closed cells to open cells being controlled within a preferred range. In this way, the heat retention and/or insulation capabilities of the material are precisely controlled as well as the ability of the material to transmit oxygen from the surrounding, ambient air directly to the location of the heat delivery patch or exothermic pad.

In accordance with the embodiment of present invention wherein heat delivery is employed, the temperature delivered directly to the nail or skin surface by the exothermic pad or heat delivery patch is precisely controlled in a narrow range, for a predetermined length of time, due to the insulation provided by the holding system. In addition, oxygen is circulated continuously through the holding system to provide proper operation of the exothermic pad/patch as well as provide air circulation to the surface of the nail or skin being treated.

It will must be seen that the object set forth above, among those made apparent from the preceding description, are efficiently obtained and, since certain changes may be made in carrying out the above process, in the described product, and in the constructions set forth without departing from the scope of the invention, it is intended that all matter contained in the above description shall be interpreted as illustrative and not in a limiting sense.

It is also to be understood that the following claims are intended to cover all of the generic and specific features of the invention herein described, and all statements of the scope of the invention which, as a matter of language, might be said to fall therebetween.

Particularly, it is to be understood that in said claims, ingredients or compounds recited in the singular are intended to include compatible mixtures of such ingredients whenever the sense permits.

Having described our invention, what we claim as new and desired to secure by Letters Patent is:

1. A film forming treatment composition for localized, topical application on humans for providing transdermal delivery of a desired therapeutic agent, said composition comprising
A. between about 0.5% and 25% by weight/volume based upon the weight/volume of the entire composition of film former;
B. between about 0.5% and 25% by weight/volume based upon the weight/volume of the entire composition of a plasticizer;
C. between about 0.5% and 20% by weight/volume based upon the weight/volume of the entire composition of urea;
D. between about 40% and 90% by weight/volume based upon the weight/volume of the entire composition of a solvent/volatile carrier; and
E. an effective amount of at least one therapeutic substance;
whereby a film forming treatment composition is attained which is capable of being applied to any desired area of an individual for providing localized, topical treatment for particular, specific diseases.

2. The treatment composition defined in claim 1, wherein said film former is further defined as comprising one selected from the group consisting of polyvinyl acetate, mixed polymers of vinyl acetate and acrylic acid, mixed polymers of acrylic acids and acrylate esters, polyvinyl acetate, polyvinyl butyryl, polyvinyl alcohols, cellulose derivatives, cellulose acetate phthalate, cellulose acetate butyrate, cellulose acetate propionate, cellulose nitrate, cellulose sulfate, ethylcellulose, and cellulose acetate.

3. The treatment composition defined in claim 1, wherein said film former is further defined as comprising ammonio methacrylate copolymer.

4. The treatment composition defined in claim 1, wherein said plasticizer is further defined as comprising at least one selected from the group consisting of triacetin, polyhydric alcohols, propylene glycol, butylene glycol, castor oil, camphor and phthalates.

5. The treatment composition defined in claim 4, wherein said plasticizer is further defined as comprising dibutyl phthalate.

6. The treatment composition defined in claim 1, wherein said solvent/volatile carrier comprises at least one selected from the group consisting of alcohols, ethanols, ketones, acetones ethers, chloroforms, aromatic hydrocarbons, toluenes, esters, ethyl acetate, and water.

7. The treatment composition defined in claim 1, wherein said solvent/volatile carrier comprises an evaporation rate which allows the complete application of the active ingredients of the composition to the skin surface while also preventing the composition from being rubbed off when the treated surface comes into contact with another surface.

8. The treatment composition defined in claim 7, wherein the solvent/volatile carrier is further defined as comprising 50% by weight/volume of acetone intermixed with 50% by weight/volume of ethanol.

9. The treatment composition defined in claim 1, wherein the therapeutic agent comprises one or more selected from the group consisting of corticosteroids, chemotherapeutic agents, anesthetics, antihistamines, anti-bacterials, anti-parasitics, anti-viral agents, anti-oxidants, tar, anthralines, immunomodulators, keratolytics, and anti-neoplastics.

10. The treatment composition defined in claim 9, wherein the therapeutic agent is further defined as comprising one or more selected from the group consisting of hydrocortisone, triamcinolone, betamethasone, 5FU, bleomycin, methotrexate, cytotoxic agents, lidocaine, prilocaine, diphenhydramine and its salts, doxepin, gentamicin, tetracycline, erythromycin, clindamycin, metronidazole, permethrin, crotamiton, acyclovir, ascorbic acid, tocopherol, imiquimod, beta glucan, salicylic acid, cytotoxic agents and immunomodulators.

11. The treatment composition defined in claim 1, wherein said composition further comprises an enhancing agent for assisting in the delivery of the therapeutic substance.

12. The treatment composition defined in claim 11, wherein said composition is further defined as comprising heat delivery means constructed for cooperative, overlying engagement therewith for providing a heat gradient to the use and delivery of said treatment composition.

13. The treatment composition defined in claim 12, wherein said enhancing agent comprises one or more selected from the group consisting of solvents, surfactants, ethers, esters, fatty acid glycerides, urea, oleates, liposomes, retinoids, and occlusive compounds.

14. A treatment system for providing the effective transdermal delivery of therapeutic agents to humans for treating a wide variety of medical conditions, said system comprising:
1. A film forming composition comprising:
1. between about 0.5% and 25% by weight/volume based upon the weight/volume of the entire composition of film former;
2. between about 0.5% and 25% by weight/volume based upon the weight/volume of the entire composition of a plasticizer;
3. between about 0.5% and 20% by weight/volume based upon the weight/volume of the entire composition of urea;
4. between about 40% and 90% by weight/volume based upon the weight/volume of the entire composition of a solvent/volatile carrier; and
5. an effective amount of at least one therapeutic substance;
B. a heat generating pad incorporating heat producing means contained therein and constructed for delivering the desired level of heat upon activation; and
C. a holding and supporting member constructed for
1. cooperating with the heat generating pad for enabling the application of heat directly to a desired application site of the film forming composition; and
2. being securely retained on a portion of the human body in overlying engagement with the heat delivery patch/pad and the film forming composition;
whereby a treatment system is realized which is capable of being secured to any desired part of the human body to provide therapeutic agents and heat to any desired site in a precisely controlled manner.

15. The treatment system defined in claim 14 wherein the holding member is constructed for mounting securement to one body part selected from the group consisting of fmgers, arms, elbows, toes, feet, legs, wrists, ankles, and the upper torso.

16. The treatment system defined in claim 14, wherein said holding and supporting member comprises an elongated, substantially planar construction incorporating fastening means for securing said member in any desired location.

17. The treatment system defined in claim 16, wherein said fastening means is defmed as comprising one selected from the group consisting of adhesives and hook and loop fasteners.

18. The treatment system defined in claim 14, wherein said holding and support member is further defined as being formed from thermoplastic elastomeric materials.

19. The treatment system defined in claim 18, wherein said thermoplastic elastomeric material is defined as comprising one selected from the group consisting of polyurethanes, polyolefins, polybutylenes, polyethylenes, polyesters, ethylene-propylene rubbers, polypropylenes, silicones, and vinyl-based resins.

20. The treatment system defined in claim 17, wherein said fastening means are defined as comprising at least two cooperating, interlocking members affixed to opposed surfaces of the holding and supporting member and positioned for mating, cooperating, interengagement with each other for securely maintaining the holding and supporting member in any desired position or location on an individual.

21. The treatment system defined in claim 20, wherein said pair of cooperating, interlocking members are further defined as comprising hook and loop fastening means with the first of said pair of interlocking members being mounted on a first surface of the holding and supporting member in spaced, cooperating relationship with the heat generating pad mounted thereto, and a second interlocking hook and loop fastening member being securely affixed to substantially the entire opposed surface of the holding and supporting member whereby cooperating, locked mounted interengagement of said first and second interlocking members is quickly and easily attained at virtually any desired location or position.

22. The treatment system defined in claim 17, wherein said mounting means is further defined as comprising an adhesive layer affixed to one surface of the holding and supporting member in association with the heat generating pad for enabling the fastening means. to securely affix the holding and supporting member and heat generating pad to any desired position or location on the skin of the individual.

23. The treatment system defined in claim 17, wherein said heat generating pad is further defined as being activated by exposure to oxygen and said treatment system is further defined as being packaged in an oxygen free, peripherally surrounding releasable container, thereby enabling a single heat generating pad to be reusably employed for short time periods by storing the delivery system in the releasable container when not in use.

24. The treatment system defined in claim 14, wherein said film former is further defined as comprising one selected from the group consisting of polyvinyl acetate, mixed polymers of vinyl acetate and acrylic acid, mixed polymers of acrylic acids and acrylate esters, polyvinyl acetate, polyvinyl butyryl, polyvinyl alcohols, cellulose derivatives, cellulose acetate phthalate, cellulose acetate butyrate, cellulose acetate propionate, cellulose nitrate, cellulose sulfate, ethylcellulose, and cellulose acetate.

25. The treatment system defined in claim 24, wherein said plasticizer is further defined as comprising at least one selected from the group consisting of triacetin, polyhydric alcohols, propylene glycol, butylene glycol, castor oil, camphor and phthalates.

26. The treatment system defined in claim 25, wherein said solvent/volatile carrier comprises at least one selected from the group consisting of alcohols, ethanols, ketones, acetones ethers, chloroforms, aromatic hydrocarbons, toluenes, esters, ethyl acetate, and water.

27. The treatment system defined in claim 26, wherein the therapeutic agent comprises one or more selected from the group consisting of corti-costeroids, chemotherapeutic agents, anesthetics, antihistamines, anti-infectives, anti-fungals, anti-bacterials, anti-parasitics, anti-viral agents, anti-oxidants, tar, anthralines, immunomodulators, keratolytics, and anti-neoplastics

28. The treatment system defined in claim 14, wherein said film forming composition further comprises an enhancing agent for assisting in the delivery of the therapeutic substance.

29. The treatment system defined in claim 28, wherein said enhancing agent comprises one or more selected from the group consisting of solvents, surfactants, ethers, esters, fatty acid glycerides, urea, oleates, liposomes, retinoids, and occlusive compounds.
